(19) United States
(12) Patent Application Publication
(10) Pub. No.: US 2022/0362111 A1
(43) Pub. Date: Nov. 17, 2022

(54) OIL POWDER FOUNDATION

(71) Applicant: Tombow Pencil Co., Ltd., Tokyo (JP)

(72) Inventors: Satoshi TATEMICHI, Tokyo (JP); Noriaki OZAKI, Tokyo (JP); Yoshikazu SOYAMA, Tokyo (JP)

(21) Appl. No.: 17/741,844

(22) Filed: May 11, 2022

(30) Foreign Application Priority Data
May 13, 2021 (JP) 2021-081794

(51) Int. Cl. A61K 8/02; A61K 8/29; A61Q 1/12

(52) U.S. Cl. CPC A61K 8/022; A61K 8/29; A61Q 1/12; A61K 2800/412; A61K 2800/31

(57) ABSTRACT

Provided is an oil powder foundation which utilizes advantageous features of a powder foundation and an oil foundation and has solved problems that these foundations have, and which gives natural finish having high hiding power. The oil powder foundation may include (a) spherical particles, (b) titanium dioxide, (c) a film-forming agent, (d) a volatile oil agent, and (e) a surfactant. Using a urethane powder, acrylic powder, nylon powder, silicone powder, cellulose powder, silica powder, or calcium carbonate powder having an average particle diameter of 5 to 20 μm as the component (a), the oil powder foundation is prepared so that the total of the component (a) and the component (b) is contained in an amount of 35 to 55% by mass and the mass ratio of the component (a) and the component (b) is 1 to 1.5:1.5 to 1.

### TECHNICAL FIELD

The present disclosure relates to an oil powder foundation.

### INTRODUCTION

The foundations recently used include mainly a solid powder foundation as well as a solid oil foundation (inner tray, stick type). Further, there have been developed a liquid foundation and a cream foundation, which are derived from the solid oil foundation, and an emulsion liquid foundation and an emulsion cream foundation, which are obtained by incorporating water or a polyhydric alcohol into the above liquid or cream foundation.

Powder foundation generally comprises about 85 to 90% by mass of a powder and 10 to 15% by mass of an oil material, and has powdery finish and has a disadvantage in that marked makeup deterioration is caused due to sebum, and the use of a makeup primer is essential before applying the powder foundation. Especially, when used in winter, the powder foundation has powdery finish. On the other hand, oil foundation comprises 40 to 45% by mass of a powder and 55 to 60% by mass of an oil material or a wax, and has oily feeling of finish, and hence oil foundation is unlikely to be used in summer in which the weather is hot, and is more likely to be used in autumn and winter.

A foundation that is improved to achieve powdery feeling despite being an oil foundation has been put on the market, and such a foundation is a stick oil foundation having powdery feeling, which uses cyclohexasiloxane as a volatile oil agent, and which has incorporated thereinto a low-viscosity oil material, such as phenyl trimethicone or isohexadecane. However, the stick oil foundation does not have a film-forming agent incorporated, so that sweat, sebum, or the like due to the above-mentioned hot weather causes makeup deterioration. The effect of diffusing or reflecting a light striking the skin to correct pores or unevenness of the skin is simultaneously considerably lowered. The stick oil foundation is required to remove such a disadvantage, specifically, required to maintain the finish just after wearing a makeup, but there is no foundation having this function.

Liquid foundation, cream foundation, emulsion liquid foundation, and emulsion cream foundation have oily feeling of finish, which is very close to that of the oil foundation, and have no powdery feeling of finish like a commercially available powder foundation.

### PATENT LITERATURE

PTL 1: Japanese Patent No. 3664246
PTL 2: Japanese Patent No. 6112863
PTL 3: Japanese Patent No. 4606187

### SUMMARY

Under the circumstances, there is desired a foundation advantageous not only in that, despite having excellent spreadability which is a feature of an oil foundation, the foundation need not use a makeup primer, but also in that the foundation is capable of changing to feel powdery immediately after being applied and thus does not feel oily, so that it can be used throughout all four seasons. Needless to say, the function of well correcting pores or unevenness of the skin is a property that the foundation should have, and the foundation is required to maintain this function as mentioned above.

A foundation is also expected to correct pores or unevenness of the skin, generally utilizing the soft-focus function (shading effect to cause pores of the skin and the like not to be noticeable) of spherical particles or the hiding function of titanium dioxide, achieving finish as natural as possible.

The present disclosure relates to an oil powder foundation which not only has excellent spreadability that is an advantageous feature of an oil foundation but also is capable of changing to feel powdery immediately after being applied. More particularly, the present disclosure is concerned with a novel oil powder foundation which is advantageous not only in that it can correct pores and unevenness of the skin, but also in that the oil powder foundation has finish and feeling equivalent to those of a conventional powder foundation.

### Technical Problem

PTL 1 discloses a gel oil foundation which has incorporated a plate powder having a particle diameter of 1 to 12 μm and an average particle diameter of 5 to 6 μm, and a spherical powder having a particle diameter of 4 to 10 μm, wherein the mass ratio of the plate powder and the spherical powder is 1:1 to 1:4, and the amount of the spherical powder incorporated is 7 to 11% by mass, wherein the oil foundation has excellent correction effect for unevenness of the surface of the skin and excellent hiding effect for pores of the skin. However, the gel oil foundation does not have such a function that it is capable of changing to feel powdery immediately after being applied, and cannot solve a problem in that the foundation feels oily after being applied.

PTL 2 discloses an oil solid cosmetic material which comprises 40 to 60% by mass of a powder containing titanium oxide, based on the mass of the cosmetic material (with the proviso that a spherical powder is contained in an amount of 0 to 10% by mass, based on the total mass of the powder), a film-forming agent, a volatile oil agent, and 2 to 12% by mass of an ester oil having a molecular weight of 500 to 1,000 and an 10B value of 0.2 or less. However, the amount of the spherical powder is 0 to 10% by mass of the total mass of the powder, and even the maximum amount of the spherical powder incorporated corresponds to as small an amount as 6% by mass of the mass of the cosmetic material, and the cosmetic material has finish and texture that cannot feel powdery at all.

PTL 3 discloses a cosmetic material for hiding sulcus cutis, which has incorporated spherical particles in an amount of 10 to 70% by mass, an oil agent in an amount of 20 to 80% by mass, and a powder other than the spherical particles. With respect to the cosmetic material, particularly, when the amount of the spherical particles incorporated is 20% by mass or less and the amount of the oil agent contained is 20% by mass or more, characteristic features, such as a uniform makeup film, smooth feeling of use, and hiding sulcus cutis, can be obtained. However, the cosmetic material does not contain a film-forming agent, and therefore fixing the powder onto the skin including sulcus cutis is difficult, so that the cosmetic material applied feels oily or has oily finish. Meanwhile, when containing the spherical particles in an amount as large as about 70% by mass, the cosmetic material has a problem in that marked makeup deterioration due to sebum is caused.

A task of the present disclosure is to provide an oil powder foundation which utilizes advantageous features that a conventional powder foundation and an oil foundation individually have and has solved problems that the powder foundation and oil foundation individually have, and which is capable of changing to feel powdery immediately after being applied, giving natural finish having high hiding power free of oily feeling.

### Solution to Problem

The present inventors have conducted extensive and intensive studies with a view toward solving the above-mentioned problems. As a result, it has been found that an oil powder foundation which comprises (a) spherical particles, (b) titanium dioxide, (c) a film-forming agent, (d) a volatile oil agent, and (e) a surfactant is an excellent oil powder foundation advantageous not only in that it has excellent spreadability that is an advantageous feature of an oil foundation, but also in that it is capable of changing to feel powdery and finish like a powder foundation immediately after being applied, enabling easy makeup without a need of a makeup primer and use throughout all four seasons.

Specifically, in the present disclosure, there is provided an oil powder foundation comprising the following components (a) to (e):
(a) spherical particles,
(b) titanium dioxide,
(c) a film-forming agent,
(d) a volatile oil agent, and
(e) a surfactant.

### Advantageous Effects

The oil powder foundation of the present disclosure has a covering effect for pores and unevenness of the surface of the skin, which is equivalent to that of a powder foundation and an oil foundation. In addition, the oil powder foundation of the present disclosure does not have a disadvantage of a powder foundation such that the finish is powdery and marked makeup deterioration due to sebum is caused. Further, the oil powder foundation of the present disclosure need not use a makeup primer for preventing makeup deterioration due to sweat or sebum. The oil powder foundation of the present disclosure is capable of changing to feel powdery immediately after being applied, and hence, needless to say, the oil powder foundation of the present disclosure does not feel oily like an oil foundation, and thus is suitable for the use in summer.

### DETAILED DESCRIPTION

From the viewpoint of the feeling of use, finish, and soft-focus effect, the spherical particles as the component (a) used in the present disclosure preferably have a volume-weighted mean diameter of 5 to 20 μm, more preferably 7 to 18 μm, further preferably 12 to 18 μm. With respect to the spherical particles as the component (a), one type of or a combination of two or more types of the spherical particles can be used.

The volume-weighted mean diameter of the particles is measured by a laser diffraction/scattering particle size distribution measurement apparatus. In the present disclosure, the volume-weighted mean diameter is a 50% median diameter which is an average particle diameter in terms of a volume.

The material for the spherical particles is preferably selected from the viewpoint of the dispersibility in the recipe, i.e., the wetting properties of the powder and from the viewpoint of the usability. As the spherical particles, for example, a urethane powder, an acrylic powder, a nylon powder, a silicone powder, a cellulose powder, a silica powder, or a calcium carbonate powder can be used. From the viewpoint of imparting soft feeling, preferred are a urethane powder, an acrylic powder, a nylon powder, and a silicone powder, which are organic polymer particles, and, from the viewpoint of the usability, a urethane powder is more preferred.

With respect to the spherical particles, a commercially available product can be used. As examples of urethane powders, there can be mentioned a true-spherical crosslinked urethane powder, such as TP Powder D-400, D-800 (each of which is manufactured by Toshiki Pigment Co., Ltd.), and DAIMICBEAZ CM-1077, CM-1157 (each of which is manufactured by Dainichiseika Color & Chemicals Mfg. Co., Ltd.).

As examples of acrylic powders, there can be mentioned polyalkyl methacrylate fine particles, such as Microsphere M (polymethyl methacrylate; spherical shape having a micro-uneven surface), Microsphere M-100 (polymethyl methacrylate; spherical shape having a smooth surface), and Microsphere M-306 (methyl methacrylate crosspolymer; spherical shape having a smooth surface) (each of which is manufactured by Matsumoto Yushi-Seiyaku Co., Ltd.).

As examples of nylon powders, there can be mentioned true-spherical nylon fine particles, such as SP-500, SP-10 (each of which is manufactured by Toray Industries Inc.).

As examples of silicone powders, there can be mentioned a silicone composite powder having a silicone resin or silicone rubber surface coated with a silicone resin, such as KMP-591, KSP-100, KSP-101, KSP-411, KSP-441 (each of which is manufactured by Shin-Etsu Chemical Co., Ltd.).

As examples of cellulose powders, there can be mentioned CELLULOBEADS D-10, CELLULOBEADS D-5 (manufactured by Daito Kasei Kogyo Co., Ltd.), and GE-800 (manufactured by Toshiki Pigment Co., Ltd.).

As examples of silica powders, there can be mentioned Silica beads SB-700 (manufactured by Miyoshi Kasei, Inc.).

As examples of calcium carbonate powders, there can be mentioned Calmaru SCS-M5 (manufactured by Sakai Chemical Industry Co., Ltd.), and NL-QC10 (manufactured by New Lime Co., Ltd.).

With respect to the spherical particles as the component (a), true-spherical particles are preferred from the viewpoint of the slip properties. The term “true-spherical” means a structure such that a powder does not have a number of small voids inside or in the surface thereof.

With respect to the titanium dioxide as the component (b) used in the present disclosure, the shape and particle diameter are not limited, but, from the viewpoint of excellent hiding power, the titanium dioxide preferably has a primary particle diameter of 0.15 to 0.3 μm. The crystal form of the titanium dioxide is preferably rutile or anatase. As examples of the titanium dioxide, there can be mentioned MP-100 (manufactured by Tayca Corporation), JR-800 (manufactured by Tayca Corporation), CR-50 (manufactured by Ishihara Sangyo Kaisha, Ltd.), MT-500SA (manufactured by Tayca Corporation), and ST455 (manufactured by Titan Kogyo, Ltd.).

The amount of the total of the spherical particles as the component (a) and the titanium dioxide as the component (b) contained in the oil powder foundation is preferably 35 to 55% by mass, more preferably 40 to 52% by mass. The ratio (mass ratio) of the component (a) and the component (b) is preferably 1 to 1.5:1.5 to 1, more preferably 1 to 1.3:1.3 to 1, further preferably 1 to 1.2:1.2 to 1.

In the present disclosure, a powder other than the spherical particles as the component (a) and the titanium dioxide as the component (b) can also be used, and there can be mentioned powders shown below. A powder having any particle structure, for example, a plate, needle-like, or porous powder can be used, and an inorganic powder, a luster powder, a composite powder, or the like may be used.

Examples of powders include talc, mica, synthetic mica, iron-containing synthetic mica, kaolin, sericite, magnesium carbonate, aluminum silicate, magnesium silicate, zinc oxide, red iron oxide, yellow iron oxide, black iron oxide, black titanium oxide, cerium oxide, barium sulfate, ultramarine blue, Prussian blue, titanated mica, iron oxide titanated mica, muscovite, plate synthetic mica, phlogopite, lepidolite, biotite, lithia mica, plate silicic anhydride, plate hydroxyapatite, bentonite, montmorillonite, hectorite, a plate ceramic powder, plate alumina, plate boron nitride, plate iron oxide, titanium oxide-coated mica, titanium oxide-treated mica, bismuth oxychloride, titanium oxide-coated bismuth oxychloride, titanium oxide-coated talc, fish scale flake, titanium oxide-coated colored mica, aluminum, and a plate glass powder. Further, an organic pigment may be used.

With respect to almost all the powders used in the present disclosure, those which have been subjected to surface treatment are preferred from the viewpoint of the wetting properties of the powder with the oil material or volatile oil agent. The surface treatment for the powder may be any of a chemical treatment and a mechanochemical treatment. Examples of surface treatments include a silicone compound treatment, a fluorine compound treatment, an amino acid treatment, a pendant treatment, a silane coupling agent treatment, a titanium coupling agent treatment, an oil agent treatment, an amino acid treatment, an N-acylated amino acid treatment, a fatty acid treatment, a metallic soap treatment, an inorganic compound treatment, a plasma treatment, a plant-derived ester treatment, and a mechanochemical treatment.

Examples of silicone compounds include dimethylpolysiloxane, methylhydrogenpolysiloxane, trimethylsiloxysilicic acid, alkylalkoxysilane, alkyl-modified silicone, and acrylate silicone.

Examples of fluorine compounds include perfluoropolyether acid, perfluoroalkylphosphoric acid, perfluoroalkylphosphoric acid ester, perfluoroalkylalkoxysilane, and fluorine-modified silicone.

Examples of amino acids include lecithin.

Examples of N-acylated amino acids include lauroyllysine, sodium dilauramidoglutamide lysine, disodium stearoylglutamate, and sodium lauroylaspartate.

Examples of fatty acids include stearic acid and myristic acid, and examples of metallic soaps include zinc myristate.

Examples of plant-derived esters include polyglyceryl-2 tetraisostearate.

From the viewpoint of improving the oil powder foundation in spreadability and suppressing the oiliness, thus improving the uniformity of the makeup cosmetic material applied over the foundation applied, the surface treatment is preferably a hydrophobic treatment, and, among the above-mentioned treatments, a silicone compound treatment or a fluorine compound treatment is preferably used. Specifically, preferred are a methylhydrogenpolysiloxane treatment, a dimethylpolysiloxane treatment, an alkylalkoxysilane treatment, and a perfluoroalkylalkoxysilane treatment, and more preferred are a methylhydrogenpolysiloxane treatment and a dimethylpolysiloxane treatment. The hydrophobic treatment can be conducted by a general method.

The film-forming agent as the component (c) used in the present disclosure is in the form of a paste or a solid at ordinary room temperature (25° C.), and has a property that the film-forming agent is compatible with the volatile oil agent as the component (d) and the volatile oil agent volatilizes on the skin to form a film.

With respect to the film-forming agent, an oil-soluble silicone resin used in general cosmetics can be used, and examples of such silicone resins include a fluorine-modified silicone resin, trimethylsiloxysilicic acid, and an acrylic silicone resin, and one member or a combination of two or more members selected from these compounds can be used.

Further, as a film-forming agent other than the oil-soluble silicone resin, there can be used a sucrose fatty acid ester, such as a terpene resin, a rosin resin, a candelilla wax extract, or dextrin isostearate, or an oil-soluble resin, such as denatured starch. These compounds can be used individually or in combination, and may be used in combination with an oil-soluble silicone resin.

Among the film-forming agents, trimethylsiloxysilicic acid and dextrin isostearate are more preferred from the viewpoint of excellent maintaining properties for the makeup effect.

From the viewpoint of achieving uniform application of the foundation when being used, the above-mentioned fluorine-modified silicone resin, trimethylsiloxysilicic acid, or acrylic silicone resin is preferably dissolved in a solvent before being used. With respect to the solvent, from the viewpoint of the dissolving power for the film-forming agent, compatibility with the volatile solvent as the component (d), and low skin stimulation, a silicone oil is preferably used. As a silicone oil, one member or two or more members selected from methyl trimethicone, dimethylpolysiloxane (2 cs), dimethylpolysiloxane (6 cs), dimethylpolysiloxane (10 cs), decamethylcyclopentasiloxane, and octamethylcyclotetrasiloxane are preferably used, and one member or two or more members selected from methyl trimethicone, dimethylpolysiloxane (2 cs), and decamethylcyclopentasiloxane are more preferably used.

When an acrylic silicone resin is used as the film-forming agent, a hydrocarbon can be used as a solvent, and isododecane is preferably used.

With respect to the fluorine-modified silicone resin used as the film-forming agent, trifluoroalkyldimethyltrimethylsiloxysilicic acid is preferred, and there can be used a commercially available product of the resin, which is preliminarily dissolved in a solvent, such as XS66-B8226 (decamethylcyclopentasiloxane solution having a solid content of 50% by mass), XS66-B8636 (dimethylpolysiloxane (10 cs) solution having a solid content of 50% by mass) (each of which is manufactured by Momentive Performance Materials Inc.).

As a commercially available product of trimethylsiloxysilicic acid, there can be used trimethylsiloxysilicic acid which is preliminarily dissolved in a solvent, such as KF-7312T (methyl trimethicone solution having a solid content of 60% by mass), KF-7312J (decamethylcyclopentasiloxane solution having a solid content of 50% by mass), KF-7312K (dimethylpolysiloxane (6 cs) solution having a solid content of 60% by mass), KF-7312L (dimethylpolysiloxane (2 cs) solution having a solid content of 50% by mass), KF-9021 (decamethylcyclopentasiloxane solution having a solid content of 50% by mass), KF-9021 L (dimethylpolysiloxane (2 cs) solution having a solid content of 50% by mass), X21-5249 (decamethylcyclopentasiloxane solution having a solid content of 50% by mass), X21-5595 (isododecane solution having a solid content of 60% by mass) (each of which is manufactured by Shin-Etsu Chemical Co., Ltd.), SS4267 (dimethylpolysiloxane solution having a solid content of 35% by mass), SR1000 (each of which is manufactured by Momentive Performance Materials Japan LLC), BY11-018 (decamethylcyclopentasiloxane solution having a solid content of 30% by mass) (manufactured by Dow Toray Co., Ltd.), or BELSIL TMS 803 (manufactured by Wacker Chemie AG).

As a commercially available product of an acrylic silicone resin, there can be used an acrylic silicone resin which is preliminarily dissolved in a solvent, such as FA 4001CM (decamethylcyclopentasiloxane solution having a solid content of 30% by mass), FA 4002ID (isododecane solution having a solid content of 40% by mass) (each of which is manufactured by Dow Toray Co., Ltd.), or KP-545 (decamethylcyclopentasiloxane solution having a solid content of 30% by mass), KP-550 (isododecane solution having a solid content of 40% by mass), KP-545L (dimethylpolysiloxane (2 cs) solution having a solid content of 40% by mass) (each of which is manufactured by Shin-Etsu Chemical Co., Ltd.).

The amount of the film-forming agent contained is preferably 3 to 30% by mass, more preferably 4 to 20% by mass, further preferably 5 to 10% by mass, in terms of the amount of the solid of the film-forming agent. When the amount of the film-forming agent contained is in the range of from 3 to 30% by mass, in terms of the amount of the solid of the film-forming agent, the spherical particles are arranged on the surface of the finished skin, and a light striking the particles is irregularly reflected, so that the skin can feel matte and powdery. Further, the film-forming agent improves the resistance to sebum and water resistance so that the spherical particles are fixed to pores or uneven surface of the skin, preventing makeup deterioration. Accordingly, there can be obtained an oil powder foundation which not only has as excellent spreadability as an oil foundation but also is capable of changing to feel powdery immediately after being applied.

With respect to the volatile oil agent as the component (d) used in the present disclosure, there is no particular limitation as long as it is an oil agent which is volatile at ordinary room temperature (25° C.) under atmospheric pressure (1 atm.) and is generally used in cosmetics, and a hydrocarbon oil, a silicone oil, an ether oil, or the like is used. The volatile oil agent as the component (d) preferably has a boiling point of 150° C. or higher.

From the viewpoint of the dissolving power for the film-forming agent as the component (c), dispersibility of the spherical particles as the component (a) and the titanium dioxide as the component (b), usability, skin stimulation, and odor, it is preferred that one type of, or two or more types of, the volatile oil agents are used.

Examples of hydrocarbon oils include hydrocarbons, such as isododecane, isotridecane, and isohexadecane.

Examples of silicone oils include volatile silicone oils, e.g., chain polysiloxanes, such as dimethylpolysiloxane, methyl trimethicone, caprylyl methicone, and ethyltrisiloxane, and cyclic polysiloxanes, such as octamethylcyclotetrasiloxane (abbreviation: D4), decamethylcyclopentasiloxane (abbreviation: D5), and dodecamethylcyclohexasiloxane (abbreviation: D6). The chain polysiloxane may be of any type of a linear chain and a branched chain.

Examples of ether oils include fluorine-containing ether oils, such as ethyl perfluorobutyl ether.

From the viewpoint of suppressing the oiliness, the volatile oil agent preferably contains at least a silicone oil, and the silicone oil is contained in the volatile oil agent preferably in an amount of 80% by mass or more, further preferably 90% by mass or more.

The volatile oil agent includes one which is used as a solvent in the commercially available trimethylsiloxysilicic acid solution or the like used as the film-forming agent as the component (c).

From the viewpoint of obtaining the oil powder foundation which exhibits excellent spreadability when being used, and which has excellent finish, the amount or total amount of the contained volatile oil agent or agents as the component (d) is preferably 5 to 50% by mass, more preferably 10 to 40% by mass, further preferably 15 to 35% by mass, based on the mass of the oil powder foundation.

With respect to the surfactant as the component (e), from the viewpoint of improving wetting properties of the spherical particles as the component (a), the titanium dioxide as the component (b), and other powders with a medium, such as the volatile oil agent, to improve the dispersibility, one member or two or more members selected from nonionic surfactants having an HLB of 10 or less are preferred, and a nonionic surfactant having an HLB of 6 or less is more preferred.

The HLB (hydrophile-lipophile balance) is determined using the Griffin's equation. When two or more nonionic surfactants are used, the HLB value is obtained by a weighted averaging the HLB values of the individual nonionic surfactants based on the ratio of the incorporated nonionic surfactants.

Examples of the nonionic surfactants having an HLB of 10 or less include sorbitan esters, such as sorbitan monoisostearate, sorbitan momooleate, sorbitan sesquiisostearate, and sorbitan sesquioleate; diglyceryl esters, such as diglyceryl monoisostearate and diglyceryl momooleate; a sucrose fatty acid ester, a polyoxyethylene alkyl ether, and a polyoxyethylene hardened castor oil.

Alternatively, a nonionic surfactant selected from modified silicones can be used. Examples of modified silicones include ones having a silicone chain which is linear, branched, or crosslinked, such as polyether-modified silicone, polyether-alkyl co-modified silicone, polyglycerol-modified silicone, and polyglycerol-alkyl co-modified silicone.

With respect to the surfactant as the component (e), one type of or two or more types of the surfactants can be used, and, from the viewpoint of the dispersibility of powder and feeling of use, the amount of the surfactant contained is preferably 0.1 to 10% by mass, more preferably 0.5 to 5% by mass, based on the mass of the oil powder foundation.

By further incorporating a wax as a component (f) into the oil powder foundation of the present disclosure, an oil powder foundation of a solid type can be provided. When using no wax, an oil powder foundation of a liquid type or an oil powder foundation of a cream type can be provided.

When incorporating the wax as the component (f), examples of waxes include natural waxes, such as carnauba wax, candelilla wax, beeswax, Japan wax, and rice bran wax; mineral waxes, such as a polyethylene wax, a paraffin wax, a microcrystalline wax, ceresin, and a silicone wax; saturated fatty acids, such as myristic acid, palmitic acid, stearic acid, arachic acid, and behenic acid; higher fatty acid esters, such as cetyl palmitate; and higher alcohols, such as cetanol, stearyl alcohol, and behenyl alcohol.

From the viewpoint of the hardness of the oil powder foundation of a solid type, excellent spreadability of the foundation when being used, and stability of the shape of the solid foundation, the amount of the contained wax as the component (f) is preferably 0.1 to 10% by mass, more preferably 1 to 8% by mass, further preferably 2 to 6% by mass, based on the mass of the oil powder foundation.

In the oil powder foundation of the present disclosure, if necessary, organic modified bentonite can be incorporated as a component (g). Particularly, the organic modified bentonite is used for imparting an appropriate viscosity to the oil powder foundation of a liquid type or a cream type.

With respect to the organic modified bentonite as the component (g), one type of or two or more types of the organic modified bentonite can be used, and, from the viewpoint of the feeling of use and imparting a viscosity, the amount of the organic modified bentonite contained is 0 to 10% by mass, preferably 1 to 8% by mass, more preferably 2 to 6% by mass, based on the mass of the oil powder foundation.

Representative organic modified bentonite is a kind of colloidal water-containing aluminum silicate having a three-layer structure, which is obtained by modifying a clay mineral with a quaternary ammonium salt cationic surfactant. Specific examples include dimethyldistearylammonium hectorite (disteardimonium hectorite), dimethylalkylammonium hectorite, benzyldimethylstearylammonium hectorite, and distearyldimethylammonium chloride-treated aluminum magnesium silicate. As a commercially available product, preferred are BENTONE 27 (benzyldimethylstearylammonium chloride-treated hectorite; manufactured by Elementis Japan) and BENTONE 38 (distearyldimethylammonium chloride-treated hectorite; manufactured by Elementis Japan).

With respect to the composition of the oil powder foundation of the present disclosure containing the above-mentioned components (a) to (g), it is preferred that the oil powder foundation contains no water.

With respect to the composition of the oil powder foundation of the present disclosure, at least one member selected from water and a polyhydric alcohol as a component (h) may be further incorporated into the oil powder foundation. In this case, the oil powder foundation of a W/O emulsion type containing a skincare component insoluble in an oil medium can be obtained. The amounts of the water and polyhydric alcohol contained are not limited, but the amount of the water contained is advantageously 1 to 30% by mass, based on the mass of the oil powder foundation, and the amount of the polyhydric alcohol contained is advantageously 0.1 to 10% by mass, based on the mass of the oil powder foundation.

In the oil powder foundation of the present disclosure, in addition to the above-mentioned components (a) to (h), various additives generally incorporated into cosmetics can be incorporated in such an amount that the effects of the present disclosure are not sacrificed, and examples of additives include an antiseptic agent, an ultraviolet light absorber, an ultraviolet light scattering agent, an oil agent, a thickener, a moisture retention agent, an antioxidant, a chelating agent, a neutralizing agent, a pH adjustor, an insect repellent, a physiologically active component, a fluorine compound, a perfume, and a salt.

As an antiseptic agent, methylparaben, ethylparaben, sodium dehydroacetate, or the like can be used. Examples of such antiseptic agents include MEKKINS M, MEKKINS E, manufactured by Ueno Fine Chemicals Industry, Ltd., and Geogard 111S, manufactured by Lonza Japan.

As an ultraviolet light absorber, PARSOL MCX or PARSOL 1789 (manufactured by DSM) can be used, and, as an ultraviolet light scattering agent, there can be used ultrafine particle titanium dioxide, such as MT-100TV, MTY-02 (manufactured by Tayca Corporation), or STR-100A-LP, STR-100C-LP, STR-100W-LP (manufactured by Sakai Chemical Industry Co., Ltd.), or fine particle zinc oxide, such as FINEX-50, NANOFINE-50LP (manufactured by Sakai Chemical Industry Co., Ltd.).

### EXAMPLES

Hereinbelow, the present disclosure will be described in more detail with reference to the following Examples, which should not be construed as limiting the scope of the present disclosure. In the following Examples, the amount of each component contained is indicated by “% by mass”.

The evaluation method and the criteria for evaluation are as follows.

An actual use test in which N=6 persons was individually conducted, and evaluation was made in respect of the degree of spreadability, powdery feeling, finish, and long-lasting property.

With respect to the criteria for evaluation, from the points of evaluation on the below-shown scale of 1 to 5 which is the maximum number of points, an average of the points of evaluation (which was rounded to one decimal) was evaluated in accordance with the four criteria shown below.

### (Scale for Points of Evaluation)

5 Points: Very excellent
4 Points: Excellent
3 Points: Intermediate
2 Points: Slightly poor
1 Point: Poor

### (Criteria for Evaluation)

⊙: An average of the points of evaluation is 4.1 or more.
◯: An average of the points of evaluation is 3.1 to 4.0.
Δ: An average of the points of evaluation is 2.1 to 3.0.
×: An average of the points of evaluation is 2.0 or less.

### Examples 1 to 5 and Comparative Example 1

The particle diameter (volume-weighted mean diameter) of the spherical particles as the component (a) was studied. The results of the evaluation made in the case where five types of particle diameters were selected are shown in Table 1.

With respect to the oil powder foundations of a solid type in Examples 1 to 5 and the foundation in Comparative Example 1, each sample was prepared as described below. Specifically, among the components shown in Table 1, the components (c) to (f) and oil agent in the respective predetermined amounts were weighed and heated to 80 to 90° C. so that they were dissolved. Then, the components (a) and (b) and coloring material in the respective predetermined amounts were added, and the resultant mixture was satisfactorily dispersed by stirring, and then allowed to stand at 80 to 90° C. so that the mixture was deaerated, and then a container was filled with the mixture, followed by solidification by cooling.

The results of the evaluation made for the samples are shown in Table 1.

From Table 1, it was found that the oil powder foundation containing spherical particles having a particle diameter of 5 to 20 μm (specifically, 7 μm, 8 μm, or 15 μm) had excellent spreadability, powdery feeling, finish, and long-lasting property. On the other hand, the oil powder foundation containing spherical particles having a particle diameter of 3 μm had slightly poor evaluation results in respect of spreadability and finish, and the oil powder foundation containing spherical particles having a particle diameter of 30 μm had slightly poor evaluation results in respect of powdery feeling and finish. In contrast, the foundation in Comparative Example 1, which does not contain a film-forming agent and a volatile solvent, had poor evaluation results in respect of finish and long-lasting property.

TABLE 1

| | | | | | | | Comparative |
|---|---|---|---|---|---|---|---|
| | | Example | | | | | Example |
| Component (% By mass) | | 1 | 2 | 3 | 4 | 5 | 1 |
| a | Acrylic powder (8 μm) *1 | 17.0 | — | — | — | — | 17.0 |
| | Urethane powder (15 μm) *2 | — | 17.0 | — | — | — | — |
| | Urethane powder (7 μm) *3 | — | — | — | — | 17.0 | — |
| | Silica beads (3 μm) *4 | — | — | 17.0 | — | — | — |
| | Silica beads (30 μm) *5 | — | — | — | 17.0 | — | — |
| b | Silicone-treated titanium oxide *6 | 20.0 | 20.0 | 20.0 | 20.0 | 20.0 | 20.0 |
| Coloring | Silicone-treated yellow iron oxide *7 | 3.0 | 3.0 | 3.0 | 3.0 | 3.0 | 3.0 |
| material | Silicone-treated red iron oxide *8 | 1.0 | 1.0 | 1.0 | 1.0 | 1.0 | 1.0 |
| | Silicone-treated black iron oxide *9 | 0.1 | 0.1 | 0.1 | 0.1 | 0.1 | 0.1 |
| c | Trimethylsiloxysilicic acid/methyl | 15.0 | 15.0 | 15.0 | 15.0 | 15.0 | — |
| | trimethicone mixture *10 | | | | | | |
| d | Isododecane *11 | 29.9 | 29.9 | 29.9 | 29.9 | — | — |
| d | Decamethylcyclopentasiloxane *12 | — | — | — | — | 29.9 | — |
| e | Sorbitan sesquioleate *13 | 1.0 | 1.0 | 1.0 | 1.0 | 1.0 | 1.0 |
| f | Carnauba wax *14 | 1.0 | 1.0 | 1.0 | 1.0 | 1.0 | 1.0 |
| f | Ceresin *15 | 4.0 | 4.0 | 4.0 | 4.0 | 4.0 | 4.0 |
| f | Polyethylene/microcrystalline wax | 3.0 | 3.0 | 3.0 | 3.0 | 3.0 | 3.0 |
| | mixture *16 | | | | | | |
| Oil agent | Squalane *17 | 5.0 | 5.0 | 5.0 | 5.0 | 5.0 | 49.9 |
| Results of | Spreadability | ⊙ | ⊙ | Δ | ⊙ | ⊙ | ⊙ |
| evaluation | Powdery feeling | ⊙ | ⊙ | ⊙ | Δ | ⊙ | Δ |
| | Finish | ⊙ | ⊙ | Δ | Δ | ⊙ | X |
| | Long-lasting property | ⊙ | ⊙ | ◯ | Δ | ⊙ | X |

*1: Polymethyl methacrylate; Matsumoto Microsphere M-100 (volume-weighted mean diameter: 8 μm), manufactured by Matsumoto Yushi-Seiyaku Co., Ltd.
*2: Polyurethane beads; CM-1157 (volume-weighted mean diameter: 15 μm), manufactured by Dainichiseika Color & Chemicals Mfg. Co., Ltd.
*3: Polyurethane beads; CM-1077 (volume-weighted mean diameter: 7 μm), manufactured by Dainichiseika Color & Chemicals Mfg. Co., Ltd.
*4: Silica beads; Godd Ball G-6C (volume-weighted mean diameter: 3 μm), manufactured by Suzuki yushi Industrial Corporation
*5: Silica beads; Godd Ball E-90C (volume-weighted mean diameter: 30 μm), manufactured by Suzuki yushi Industrial Corporation
*6: Silicone-treated titanium oxide; SA-Titanium CR-50 (100%), manufactured by Miyoshi Kasei, Inc.
*7: Silicone-treated yellow iron oxide; SA-Yellow LL-100P (100%), manufactured by Miyoshi Kasei, Inc.
*8: Silicone-treated red iron oxide; SA-Red R-516PS (100%), manufactured by Miyoshi Kasei, Inc.
*9: Silicone-treated black iron oxide; SA-Black BL-100P (100%), manufactured by Miyoshi Kasei, Inc.
*10: Trimethylsiloxysilicic acid (methyl trimethicone solution having a solid content of 60% by mass); KF-7312T, manufactured by Shin-Etsu Chemical Co., Ltd.
*11: MARUKAZOL R, manufactured by Maruzen Petrochemical Co., Ltd.
*12: DOWSILTM SH 245 Fluid, manufactured by Dow Toray Co., Ltd.
*13: RHEODOL AO-15V, manufactured by Kao Corporation
*14: Carnauba wax No. 1, manufactured by S. KATO & CO.
*15: Refined Ceresin N, manufactured by Nikko Rica Corporation
*16: PMWAX 82, manufactured by Nikko Rica Corporation
*17: Sugar squalane, manufactured by Nikko Chemicals Co., Ltd.

### Examples 6 to 13

With respect to the oil powder foundation of a solid type, the amount of the contained spherical particles as the component (a) and the amount of the contained titanium dioxide as the component (b) were studied. The types and amounts of the components contained are shown in Table 2. The oil powder foundation was prepared in the same manner as in Example 1.

As apparent from Table 2, the oil powder foundations in Examples 6 to 11, in which the ratio of the spherical particles and titanium dioxide incorporated is spherical particles: titanium dioxide=1.5:1 to 1:1.5 (mass ratio), and the amount of the total of the spherical particles and titanium dioxide contained is 35 to 55% by mass, had totally excellent results.

The oil powder foundation in Example 12, in which the amount of the total of the spherical particles and titanium dioxide contained is less than 35% by mass, had slightly poor evaluation results in respect of powdery feeling, finish, and long-lasting property, and the oil powder foundation in Example 13, in which the amount of the total of the spherical particles and titanium dioxide contained is more than 55%, had slightly poor evaluation results in respect of long-lasting property.

TABLE 2

| | | Example | | | | | | | |
|---|---|---|---|---|---|---|---|---|---|
| Component (% By mass) | | 6 | 7 | 8 | 9 | 10 | 11 | 12 | 13 |
| a | Acrylic powder (8 μm) | 21.0 | 14.0 | 33.0 | 22.0 | 24.0 | 27.0 | 18.0 | 34.2 |
| b | Silicone-treated titanium oxide | 14.0 | 21.0 | 22.0 | 33.0 | 16.0 | 18.0 | 12.0 | 22.8 |
| Coloring | Silicone-treated yellow iron oxide | 3.0 | 3.0 | 3.0 | 3.0 | 3.0 | 3.0 | 3.0 | 3.0 |
| material | Silicone-treated red iron oxide | 1.0 | 1.0 | 1.0 | 1.0 | 1.0 | 1.0 | 1.0 | 1.0 |
| | Silicone-treated black iron oxide | 0.1 | 0.1 | 0.1 | 0.1 | 0.1 | 0.1 | 0.1 | 0.1 |
| c | Trimethylsiloxysilicic acid/methyl | 15.0 | 15.0 | 15.0 | 15.0 | 15.0 | 15.0 | 15.0 | 15.0 |
| | trimethicone mixture | | | | | | | | |
| d | Isododecane | 31.9 | 31.9 | 11.9 | 11.9 | 26.9 | 21.9 | 36.9 | 9.9 |
| e | Sorbitan sesquioleate | 1.0 | 1.0 | 1.0 | 1.0 | 1.0 | 1.0 | 1.0 | 1.0 |
| f | Carnauba wax | 1.0 | 1.0 | 1.0 | 1.0 | 1.0 | 1.0 | 1.0 | 1.0 |
| f | Ceresin | 4.0 | 4.0 | 4.0 | 4.0 | 4.0 | 4.0 | 4.0 | 4.0 |
| f | Polyethylene/microcrystalline wax | 3.0 | 3.0 | 3.0 | 3.0 | 3.0 | 3.0 | 3.0 | 3.0 |
| | mixture | | | | | | | | |
| Oil agent | Squalane | 5.0 | 5.0 | 5.0 | 5.0 | 5.0 | 5.0 | 5.0 | 5.0 |
| Amount of spherical particles + titanium oxide | | 35 | 35 | 55 | 55 | 40 | 45 | 30 | 57 |
| Spherical particles:titanium oxide mass ratio | | 1.5:1 | 1:1.5 | 1.5:1 | 1:1.5 | 1.5:1 | 1.5:1 | 1.5:1 | 1.5:1 |
| Results of | Spreadability | ⊙ | ⊙ | ◯ | ◯ | ⊙ | ⊙ | ⊙ | ⊙ |
| evaluation | Powdery feeling | ◯ | ⊙ | ⊙ | ⊙ | ⊙ | ⊙ | Δ | ⊙ |
| | Finish | ⊙ | ◯ | ⊙ | ⊙ | ⊙ | ⊙ | Δ | ⊙ |
| | Long-lasting property | ⊙ | ⊙ | ⊙ | ⊙ | ⊙ | ⊙ | Δ | Δ |

### Examples 14 to 16 and Comparative Example 2

With respect to the oil powder foundation of a solid type, the amount of the contained film-forming agent as the component (c) was studied. The types and amounts of the components contained are shown in Table 3. The oil powder foundation was prepared in the same manner as in Example 1.

From Table 3, it was found that the oil powder foundations in Examples 14 and 16, in which the amount of the film-forming agent contained is 9% by mass, in terms of the amount of the solid of the film-forming agent, had excellent spreadability, powdery feeling, finish, and long-lasting property. The oil powder foundation in Example 15, in which the amount of the film-forming agent contained is 31.8% by mass, in terms of the amount of the solid of the film-forming agent, had poor spreadability and slightly poor evaluation results in respect of powdery feeling. In contrast, the foundation in Comparative Example 2, which does not contain a film-forming agent as the component (c), had poor evaluation results in respect of long-lasting property.

TABLE 3

| | | | | | Comparative |
|---|---|---|---|---|---|
| | | Example | Example | Example | Example |
| Component (% By mass) | | 14 | 15 | 16 | 2 |
| a | Acrylic powder (8 μm) | 25.0 | 15.0 | 25.0 | 25.0 |
| b | Silicone-treated titanium | 20.0 | 20.0 | 20.0 | 20.0 |
| | oxide | | | | |
| Coloring | Silicone-treated yellow | 3.0 | 3.0 | 3.0 | 3.0 |
| material | iron oxide | | | | |
| | Silicone-treated red | 1.0 | 1.0 | 1.0 | 1.0 |
| | iron oxide | | | | |
| | Silicone-treated black | 0.1 | 0.1 | 0.1 | 0.1 |
| | iron oxide | | | | |
| c | Trimethylsiloxysilicic | 15.0 | 53.0 | — | — |
| | acid/methyl trimethicone | | | | |
| | mixture | | | | |
| c | Dextrin isostearate *25 | — | — | 9.0 | — |
| d | Isododecane | 21.9 | 1.9 | 27.9 | 36.9 |
| e | Sorbitan sesquioleate | 1.0 | 1.0 | 1.0 | 1.0 |
| f | Carnauba wax | 1.0 | 1.0 | 1.0 | 1.0 |
| f | Ceresin | 4.0 | 4.0 | 4.0 | 4.0 |
| f | Polyethylene/ | 3.0 | — | 3.0 | 3.0 |
| | microcrystalline wax | | | | |
| | mixture | | | | |
| Oil agent | Squalane | 5.0 | — | 5.0 | 5.0 |
| Results of | Spreadability | ⊙ | Δ | ⊙ | ⊙ |
| evaluation | Powdery feeling | ⊙ | Δ | ⊙ | ⊙ |
| | Finish | ⊙ | ⊙ | ⊙ | ⊙ |
| | Long-lasting property | ⊙ | ⊙ | ⊙ | X |

*25: UNIFILMA HVY, manufactured by Chiba Flour Milling Co., Ltd.

Other examples of recipes of the oil powder foundation of the present disclosure are described below.

Recipe examples of an oil powder foundation of a liquid type are shown in Table 4.

### (Preparation Method)

A: The components (c) to (e) and (g) and oil agent are heated to 80 to 90° C. and uniformly dissolved.

B: The components (a) and (b) and coloring material are added to and uniformly dispersed in A.

C: B was deaerated, and then a container was filled with the resultant B and cooled, obtaining an oil powder foundation of a liquid type.

The oil powder foundations of a liquid type in Recipe Examples 1 and 2 exhibited excellent spreadability when being applied, and had excellent finish that feels powdery without oiliness.

TABLE 4

Recipe Example

| | | |
|---|---|---|
| Component (% By mass) | 1 | 2 |

| | | | |
|---|---|---|---|
| a | Acrylic powder (8 μm) | 24.0 | 24.0 |
| b | Silicone-treated titanium | 25.0 | 25.0 |
| | oxide | | |
| Coloring | Silicone-treated mica | 5.0 | 5.0 |
| material | Silicone-treated yellow | 3.3 | 3.3 |
| | iron oxide | | |
| | Silicone-treated red | 0.7 | 0.7 |
| | iron oxide | | |
| | Silicone-treated black | 0.1 | 0.1 |
| | iron oxide | | |
| c | Trimethylsiloxysilicic | 15.0 | 5.0 |
| | acid/methyl trimethicone | | |
| | mixture | | |
| d | Cyclohexasiloxane/ | 12.9 | 22.9 |
| | cyclopentasiloxane | | |
| | mixture *18 | | |
| Oil agent | Mineral oil *19 | 5.0 | 5.0 |
| e | Sorbitan sesquioleate | 1.0 | 1.0 |
| e | Polyether-modified | 3.0 | 3.0 |
| | silicone *20 | | |
| g | Disteardimonium | 5.0 | 5.0 |
| | hectorite *21 | | |

*18: DOWSILTM 345 Fluid, manufactured by Dow Toray Co., Ltd.
*19: MORESCO-WHITE P-40, manufactured by MORESCO Corporation
*20: KF-6017, manufactured by Shin-Etsu Chemical Co., Ltd.
*21: BENTONE 38, manufactured by Elementis Japan

Recipe examples of an oil powder foundation of a W/0 emulsion type are shown in Table 5.

### (Preparation Method)

A: The oil-phase components (c) to (e) and (g) and oil agent are heated to 80 to 90° C. and uniformly dissolved.

B: The components (a) and (b) and coloring material are added to and uniformly dispersed in A.

C: The aqueous-phase component (h) and antiseptic agent which have been preliminarily heated are added and the resultant mixture is emulsified and dispersed.

D: C was poured into a metal dish, and cooled and solidified, obtaining an oil powder foundation of a W/O emulsion type.

The oil powder foundations of a W/O emulsion type in Recipe Examples 3 and 4 exhibited appropriate spreadability and comfortable feeling of use when being applied to a wide area of the face or a part that frequently moves, such as cheek, and further had such excellent film-forming properties that excellent long-lasting property was achieved. Further, the oil powder foundations contain glycerol as a moisture retention component and therefore are effective in skincare.

TABLE 5

Recipe Example

| | | |
|---|---|---|
| Component (% By mass) | 3 | 4 |

| | | | |
|---|---|---|---|
| a | Acrylic powder (9 μm) *22 | 21.0 | 24.0 |
| b | Silicone-treated titanium | 14.0 | 25.0 |
| | oxide | | |
| Coloring | Silicone-treated mica | 5.0 | 5.0 |
| material | Silicone-treated yellow | 3.3 | 3.3 |
| | iron oxide | | |
| | Silicone-treated red | 0.7 | 0.7 |
| | iron oxide | | |
| | Silicone-treated black | 0.1 | 0.1 |
| | iron oxide | | |
| c | Trimethylsiloxysilicic | 15.0 | 5.0 |
| | acid/methyl trimethicone | | |
| | mixture | | |
| d | Cyclohexasiloxane/ | 11.8 | 10.0 |
| | cyclopentasiloxane | | |
| | mixture | | |
| Oil agent | Mineral oil | 5.0 | 2.8 |
| e | Sorbitan sesquioleate | 1.0 | 1.0 |
| e | Polyether-modified | 3.0 | 3.0 |
| | silicone | | |
| g | Disteardimonium | 5.0 | 5.0 |
| | hectorite | | |
| h | Purified water | 10.0 | 10.0 |
| h | Glycerol *23 | 5.0 | 5.0 |
| | Antiseptic agent *24 | 0.1 | 0.1 |

*22: Methyl methacrylate crosspolymer; Matsumoto Microsphere M-306 (volume-weighted mean diameter: 9 μm), manufactured by Matsumoto Yushi-Seiyaku Co., Ltd.
*23: RG CO P, manufactured by NOF Corporation
*24: MEKKINS M, manufactured by Ueno Fine Chemicals Industry, Ltd.

### INDUSTRIAL APPLICABILITY

By the present disclosure, there can be provided an oil powder foundation which is a cosmetic material that has powdery feeling at least equivalent to that of a conventional powder foundation and has solved a problem of makeup deterioration which is a disadvantage of a conventional powder foundation. The oil powder foundation of the present disclosure can be prepared in the form of a solid type, a liquid type, a cream type, or the like according to the appropriately selected recipe.

Additional aspects and features of the present disclosure are presented below, without limitation, as a series of paragraphs alphanumerically designated for clarity and efficiency. Each of these paragraphs can be combined with one or more other paragraphs, and/or with disclosure from elsewhere in this application. Some of the paragraphs below may expressly refer to and further limit other paragraphs, providing without limitation examples of some of the suitable combinations.

A0. An oil powder foundation comprising the following components (a) to (e):
(a) spherical particles,
(b) titanium dioxide,
(c) a film-forming agent,
(d) a volatile oil agent, and
(e) a surfactant.

A1. The oil powder foundation according to A0, wherein the spherical particles as the component (a) have a volume-weighted mean diameter of 5 to 20 μm.

A2. The oil powder foundation according to A0 or A1, wherein the spherical particles as the component (a) are a urethane powder, an acrylic powder, a nylon powder, a silicone powder, a cellulose powder, a silica powder, or a calcium carbonate powder.

A3. The oil powder foundation according to any one of paragraphs A0 through A2, which contains the total of the component (a) and the component (b) in an amount of 35 to 55% by mass, and the mass ratio of the component (a) and the component (b) is 1.5:1 to 1:1.5.

A4. The oil powder foundation according to any one of paragraphs A0 through A3, which contains the component (c) in an amount of 3 to 30% by mass, in terms of the amount of the solid of the component (c).

A5. The oil powder foundation according to any one of paragraphs A0 through A4, wherein the component (d) is a volatile oil agent having a boiling point of 150° C. or higher, wherein the oil powder foundation contains the component (d) in an amount of 5 to 50% by mass.

A6. The oil powder foundation according to any one of paragraphs A0 through A5, wherein the component (e) is a surfactant having an HLB of 10 or less.

A7. The oil powder foundation according to any one of paragraphs A0 through A6, further comprising a wax as a component (f).

A8. The oil powder foundation according to any one of paragraphs A0 through A7, further comprising organic modified bentonite as a component (g).

A9. The oil powder foundation according to any one of paragraphs A0 through A8, further comprising at least one member selected from water and a polyhydric alcohol as a component (h).

A10. The oil powder foundation according to any one of paragraphs A0 through A9, which contains no water.

1. An oil powder foundation comprising:
(a) spherical particles,
(b) titanium dioxide,
(c) a film-forming agent,
(d) a volatile oil agent, and
(e) a surfactant.

2. The oil powder foundation according to claim 1, wherein the spherical particles have a volume-weighted mean diameter of 5 to 20 μm.

3. The oil powder foundation according to claim 1, wherein the spherical particles comprise a urethane powder, an acrylic powder, a nylon powder, a silicone powder, a cellulose powder, a silica powder, or a calcium carbonate powder.

4. The oil powder foundation according to claim 1, wherein a total of the spherical particles and the titanium dioxide in combination are present in an amount of 35 to 55% by mass, and a mass ratio of the spherical particles and the titanium dioxide is 1.5:1 to 1:1.5.

5. The oil powder foundation according to claim 1, wherein a solid of the film-forming agent is present in an amount of 3 to 30% by mass.

6. The oil powder foundation according to claim 1, wherein the volatile oil agent has a boiling point of 150° C. or higher, and wherein the oil powder foundation contains the volatile oil agent in an amount of 5 to 50% by mass.

7. The oil powder foundation according to claim 1, wherein the surfactant has a hydrophile-lipophile balance (HLB) of 10 or less.

8. The oil powder foundation according to claim 1, further comprising a wax.

9. The oil powder foundation according to claim 1, further comprising organic modified bentonite.

10. The oil powder foundation according to claim 1, further comprising at least one member selected from water and a polyhydric alcohol.

11. The oil powder foundation according to claim 1, wherein the oil powder foundation is free of water.

12. An oil powder foundation comprising:
spherical particles having an average diameter of 5 to 20 μm;
titanium dioxide;
a film-forming agent;
a volatile oil agent; and
a surfactant
wherein the spherical particles and the titanium dioxide together amount to 35% to 55% by mass, with respect to the oil powder foundation; and
wherein a mass ratio of the spherical particles to the titanium dioxide is (1 to 1.5):(1.5 to 1).

13. The oil powder foundation of claim 12, wherein the spherical particles are selected from the group consisting of a urethane powder, an acrylic powder, a nylon powder, a silicone powder, a cellulose powder, a silica powder, and a calcium carbonate powder.

14. The oil powder foundation according to claim 12, wherein a solid of the film-forming agent is present in an amount of 3 to 30% by mass.

15. The oil powder foundation according to claim 12, wherein the volatile oil agent has a boiling point of 150° C. or higher, and wherein the oil powder foundation contains the volatile oil agent in an amount of 5 to 50% by mass.

16. The oil powder foundation according to claim 12, wherein the surfactant has a hydrophile-lipophile balance (HLB) of 10 or less.

17. The oil powder foundation according to claim 12, further comprising a wax.

18. The oil powder foundation according to claim 12, further comprising organic modified bentonite.

19. The oil powder foundation according to claim 12, further comprising at least one member selected from water and a polyhydric alcohol.

20. The oil powder foundation according to claim 12, wherein the oil powder foundation is free of water.
